(19) United States
(12) Patent Application Publication
(10) Pub. No.: US 2016/0053298 A1
(43) Pub. Date: Feb. 25, 2016

(54) METHODS FOR IDENTIFYING PROTEINS AND COMPOUNDS THAT MODULATE THE ACTIVITY OF OTUB1

(71) Applicant: The Johns Hopkins University, Baltimore, MD (US)

(72) Inventors: Cynthia Wolberger, Baltimore, MD (US); Anthony Dibello, Baltimore, MD (US); Reuven Wiener, Herzeliya (IL)

(73) Assignee: The Johns Hopkins University, Baltimore, MD (US)

(21) Appl. No.: 14/785,429

(22) PCT Filed: Apr. 14, 2014
(86) PCT No.: PCT/US2014/033947

§ 371 Date: Oct. 19, 2015

Related U.S. Application Data
(60) Provisional application No. 61/813,800, filed on Apr. 19, 2013.

(51) Int. Cl. C12Q 1/37

(52) U.S. Cl. CPC C12Q 1/37; G01N 2333/948; G01N 2333/9015; G01N 2440/36

(57) ABSTRACT

The present invention describes that OTUB1 cleavage of K48 poly-ubiquitin is stimulated by a select subset of E2 enzymes, and that this stimulation is regulated by the ubiquitin-charged state of the E2 and free ubiquitin. Structural and biochemical studies of OTUB1 and UBCH5B show that the E2 stimulates binding of the polyubiquitin substrate by contacting the OTUB1 N-terminal ubiquitin-binding helix. Methods for identifying E2 enzymes which stimulate or inhibit cleavage of K48 polyubiquitin, as well as novel target compounds which modulate this interaction are provided.

### REFERENCE TO RELATED APPLICATIONS

This application claims the benefit of U.S. Provisional Patent Application No. 61/813,800, filed on Apr. 19, 2013, which is hereby incorporated by reference for all purposes as if fully set forth herein.

### BACKGROUND OF THE INVENTION

Ubiquitination is a reversible post-translational modification that plays a pivotal role in regulating a broad range of physiological processes including proteasomal degradation, transcription, membrane trafficking and the DNA damage response. The C-terminus of ubiquitin is joined by an isopeptide linkage to lysine side chains in a three-enzyme cascade. The E1 ubiquitin activating enzyme charges the E2 ubiquitin conjugating enzyme with ubiquitin, yielding an E2 with a thioester linkage between its active site cysteine and the ubiquitin C-terminus (E2˜Ub). An E3 ubiquitin ligase then binds to both substrate and charged E2˜Ub and facilitates the transfer of ubiquitin to a substrate lysine. Substrates can be modified with a single ubiquitin or with different types of polyubiquitin chains distinguished by the particular ubiquitin lysine through which one ubiquitin is joined to the next. Polyubiquitin chains with different linkage types signal distinct outcomes 4: K48-linked polyubiquitin targets substrates to the proteasome, whereas K63-linked chains are non-degradative signals that play a role in the DNA damage response. Ubiquitination is reversed by deubiquitinating enzymes (DUBs), which remove ubiquitin from substrates as well as disassemble polyubiquitin chains. The balance between the opposing activities of ubiquitinating and deubiquitinating enzymes thus determines the levels and distribution of ubiquitination in the cell.

Ubiquitin plays a multifaceted role in orchestrating the cellular response to DNA double strand breaks (DSBs). Both monoubiquitination and polyubiquitination of substrates are needed for the ordered recruitment of downstream effectors and DNA repair. A key step is the attachment of K63-linked polyubiquitin chains at sites of DNA damage, which recruit RAP80 and the BRCA1 complex. Accumulation of K63 polyubiquitin at repair foci was unexpectedly shown to be regulated by OTUB1 11, a K48 linkage-specific deubiquitinating enzyme. OTUB1 inhibits K63 polyubiquitin chain synthesis by the E2, UBC13 (UBE2N), in a manner that does not require OTUB1 DUB activity, but that depends upon ˜40 residues N-terminal to the OTUB1 catalytic domain. Interestingly, OTUB1 also inhibits other E2 enzymes such as UBCH5A-C (UBE2D1-3) and UBCH6 (UBE2E1), although the in vivo significance of this observation is not yet known.

Structural studies of UBC13 14 and UBCH5B 15 showed that OTUB1 binds directly to the E2˜Ub thioester and holds the donor ubiquitin in its proximal ubiquitin binding site, which includes the OTUB1 N-terminal residues shown to be critical for inhibition. OTUB1 binding to the charged E2˜Ub is allosterically regulated by an additional free ubiquitin monomer that binds to the OTUB1 distal ubiquitin-binding site, triggering conformational changes in the OTU domain and in the N-terminal residues that favor binding of the UBC13˜Ub donor ubiquitin in the OTUB1 proximal site. The relative configuration of proximal and distal ubiquitins mimics K48 diubiquitin, indicating that OTUB1 isopeptidase specificity for K48 linkages as well as the allosteric communication between proximal and distal sites has been adapted for noncatalytic inhibition of E2 enzymes. The requirement for the binding of an allosteric free ubiquitin monomer to OTUB1 raised the interesting possibility that changes in nuclear ubiquitin concentrations might regulate the ability of OTUB1 to inhibit E2 enzymes.

The repressive complex formed by OTUB1 binding to charged E2˜Ub and free ubiquitin presumably interferes with OTUB1 DUB activity by competing with binding to a K485 polyubiquitin substrate. Since OTUB1 can also bind to uncharged E2 enzymes, which would not prevent binding of K48 polyubiquitin, the effect of uncharged E2 enzymes on OTUB1 DUB activity was not clear.

### SUMMARY OF THE INVENTION

In accordance with the methods of the present invention, it is found that OTUB1 DUB activity is strongly simulated by certain E2 enzymes, and that this stimulation is dependent on the concentration of free ubiquitin and the relative proportion of charged E2˜Ub thioester. The results herein indicate that the relative proportion of charged and uncharged E2s and the concentration of free ubiquitin determine the balance between OTUB1/E2 complexes that actively cleave K48 polyubiquitin chains and OTUB1/Ub/E245b complexes that inhibit both OTUB1 DUB activity and E2 ubiquitin conjugating activity. OTUB1/E2 complexes thus appear to serve a dual role in regulating levels of ubiquitin conjugation in response to fluctuations in E2 charging and available free ubiquitin.

In accordance with an embodiment, the present invention provides a method for screening compounds which modulate the activity of an E2 peptide on the isopeptidase activity of OTUB1 comprising: a) providing a solution comprising a sufficient amount of K48 UB2 OTUB1 enzyme substrate; b) adding to the solution of a) a known quantity of an E2 enzyme; c) adding to the solution of a) a known quantity of a target compound; d) contacting the solution of c) with a known amount of OTUB1 enzyme for a specified period of time; to allow the enzymatic deubiquination of the K48 UB2 by OTUB1; e) quenching the deubiquination reaction of d); f) separating the resultant reaction products from the reaction of d); g) quantifying the amount of deubiquination of the K48 UB2 and comparing the amount of deubiquination to a control solution without the E2 enzyme and a positive control solution without the target compound; and g) determining whether the amount of deubiquination was increased or decreased relative to the amount of deubiquination in the control solution and whether the amount of deubiquination was increased or decreased relative to the amount of deubiquination in the positive control solution.

In accordance with another embodiment, the present invention provides a method of screening compounds useful for the treatment ubiquination related diseases and disorders comprising: a) providing a solution comprising a sufficient amount of K48 UB2 OTUB1 enzyme substrate; b) adding to the solution of a) a known quantity of an E2 enzyme; c) adding to the solution of a) a known quantity of a target compound; d) contacting the solution of c) with a known amount of OTUB1 enzyme for a specified period of time; to allow the enzymatic deubiquination of the K48 UB2 by OTUB1; e) quenching the deubiquination reaction of d); f) separating the resultant reaction products from the reaction of d); g) quantifying the amount of deubiquination of the K48 UB2 and comparing the amount of deubiquination to a control solution without the E2 enzyme and a positive control solution without the target compound; and g) determining whether the amount of deubiquination was increased or decreased relative to the amount of deubiquination in the control solution and whether the amount of deubiquination was increased or decreased relative to the amount of deubiquination in the positive control solution.

In accordance with a further embodiment, the present invention provides a method for measuring the modulatory activity of an E2 peptide on the isopeptidase activity of OTUB1 comprising: a) providing a solution comprising a sufficient amount of K48 UB2 OTUB1 enzyme substrate; b) adding to the solution of a) a known quantity of an E2 enzyme; c) contacting the solution of b) with a known amount of OTUB1 enzyme for a specified period of time; to allow the enzymatic deubiquination of the K48 UB2 by OTUB1; d) quenching the deubiquination reaction of c); e) separating the resultant reaction products from the reaction of c); f) quantifying the amount of deubiquination of the K48 UB2 and comparing the amount of deubiquination to a control solution without the E2 enzyme; and g) determining whether the amount of deubiquination was increased or decreased relative to the amount of deubiquination in the control solution.

In accordance with still another embodiment, the present invention provides a method for measuring the isopeptidase activity of OTUB1 comprising: a) providing a solution comprising a sufficient amount of K48 UB2 OTUB1 enzyme substrate; b) contacting the solution of a) with a known amount of OTUB1 enzyme for a specified period of time; to allow the enzymatic deubiquination of the K48 UB2 by OTUB1; c) quenching the deubiquination reaction of b); d) separating the resultant reaction products from the reaction of b); and e) quantifying the amount of deubiquination of the K48 UB2.

### BRIEF DESCRIPTION OF THE DRAWINGS

FIG. 1 shows that E2 enzymes stimulate OTUB1 DUB activity. 1A. FRET-based assay for cleavage of internally quenched fluorescent K48 Ub2 (0.4 μM) by OTUB1 (0.03 μM) in the absence and presence of 11 different E2 enzymes (5 μM). Cleavage of K48 Ub2 results in an increase in fluorescence. 1B. Initial reaction velocities for the cleavage of K48 Ub2 (0.4 μM) by OTUB1 (0.03 μM) as a function of the log of the concentration of UBCH5B. Data were collected using a FRET-based deubiquitination assay and the UBCH5B EC50 value was fit using GraphPad Prism. 1C. K48 Ub2 (15 μM) cleavage by OTUB1 (0.5 μM) in the presence and absence of UBCH5B (25 μM). Samples analyzed by SDS-PAGE and Coomassie stain. 1D. Steady-state kinetic saturation curve for cleavage of K48 Ub2 by OTUB1 (0.03 μM) in the presence (red curve) and absence (blue curve) of UBCH5B (10 μM). Cleavage of unmodified K48 Ub2 was monitored by SDS-PAGE and gel densitometry.

FIG. 2 illustrates that OTUB1-E2 interactions are required for stimulation of DUB activity. 2A. FRET-based assay for cleavage of K48 Ub2 (0.4 μM) by wild type OTUB1 and OTUB1-T134R (0.03 μM) in the presence and absence of UBCH5B (5 μM). 2B. Effect of OTUB1 N-terminal deletions on stimulation by UBCH5B. Initial reaction velocities for the cleavage of K48 Ub2 by OTUB1 in the presence and absence of UBCH5B were measured using a FRET-based deubiquitination assay. For all measurements, concentrations of 0.03 μM OTUB1, 2 μM UBCH5B, and 0.4 μM K48 Ub2 were used, except in the case of the OTUB1-430 construct, for which 8 μM OTUB1 and 5 μM UBCH5B were used. 2C. Cleavage of ubiquitin-AMC (10 μM) by OTUB1 (5 μM) in the presence and absence of UBCH5B (10 μM). 2D. (Top) HeLa cells were transfected with FLAG-tagged wild-type or mutant versions of human OTUB1. 20 hours later cells were irradiated with 5 Gy, and processed for 53BP1 and FLAG immunofluorescence 4 hours post-ionizing radiation. (Bottom) Quantification of the immunofluorescence data. Data are represented as the mean±SEM (n=3). At least 100 cells were counted in each experiment.

FIG. 3 shows that UBCH5B stabilizes the OTUB1 ubiquitin-binding helix. 3A. left: Structure of hybrid human/worm OTUB1 (green) bound to ubiquitin aldehyde (Uba1, yellow), and UBCH5BC85S˜Ub in which the donor Ub (red) is covalently linked to the active-site serine (S85) of UBCH5B (blue) via an oxyester linkage. OTUB1 residues 1-21 (human numbering) are disordered. Right: 90° view. 3B. Side chain interactions between the N-terminal helix of OTUB1, the proximal donor ubiquitin, and UBCH5B. 3C. FRET-based assay for the cleavage of K48 Ub2 (0.4 μM) by wild type and mutant constructs of OTUB1 (0.03 μM) in the presence and absence of UBCH5B (5 μM). 3D. FRET-based assay for the cleavage of K48 Ub2 (0.4 μM) by OTUB1 (0.03 μM) in the presence and absence of wild type and mutant constructs of UBCH5B (5 μM).

FIG. 4 depicts the effect of free ubiquitin and E2 charging on OTUB1 DUB activity. 4A. Model for binding of UBCH5˜Ub to OTUB1 and K48 Ub2. The structure of UBCH5A˜Ub (PDB ID: 4AP4) is aligned with the OTUB1-Uba1-UBCH5B˜Ub quaternary complex. 4B. FRET-based assay for the cleavage of K48 Ub2 (0.4 μM) by OTUB1 (0.03 μM) in the presence and absence of UBCH5B or UBCH5B˜Ub (1 μM) and free ubiquitin (10 μM). 4C. Initial reaction velocities for the cleavage of K48 Ub2 (0.4 μM) by OTUB1 (0.03 μM) in the presence of UBCH5B (black circles) or UBCH5B˜Ub (0.5 μM) (black squares) as a function of the log of the concentration of free ubiquitin. IC₅₀ value was fit to the UBCH5B˜Ub data (blue curve). A fit to the UBCH5B data is shown in red. 4D. Three possible states of OTUB1-E2 complexes. Top: OTUB1 (green) bound to K48 Ub2 (red) and E2 (blue) (left) or E2 (blue)˜Ub (magenta) thioester. Bottom: OTUB1 bound to free Ub (yellow) and E2˜Ub thioester. The free ubiquitin (yellow) and the ubiquitin (magenta) conjugated to the E2 bind in place of K48 Ub2.

FIG. 5 shows the kinetic characterization of OTUB1 K48-isopeptidase activity in the absence of E2. Steady-state kinetic saturation curve for cleavage of K48 Ub2 by OTUB1 (0.03 μM). Cleavage of unmodified K48 Ub2 was monitored by SDS-PAGE and gel densitometry.

FIG. 6 depicts the electron density for the N-terminal helix of OTUB1. The OTUB1-Uba1:UBCH5B˜Ub crystal structure contained clear density for the N-terminal helix of the hybrid human/worm OTUB1 construct (green). Density for residues 1 through 21 of the N-terminal helix was not observed.

FIG. 7 depicts a structural model for activated OTUB1 bound to diubiquitin substrate. The OTUB1-Uba1:UBCH5B˜Ub crystal structure provides a model of UBCH5B-activated OTUB1 bound to a K48 Ub2 substrate.

### DETAILED DESCRIPTION OF THE INVENTION

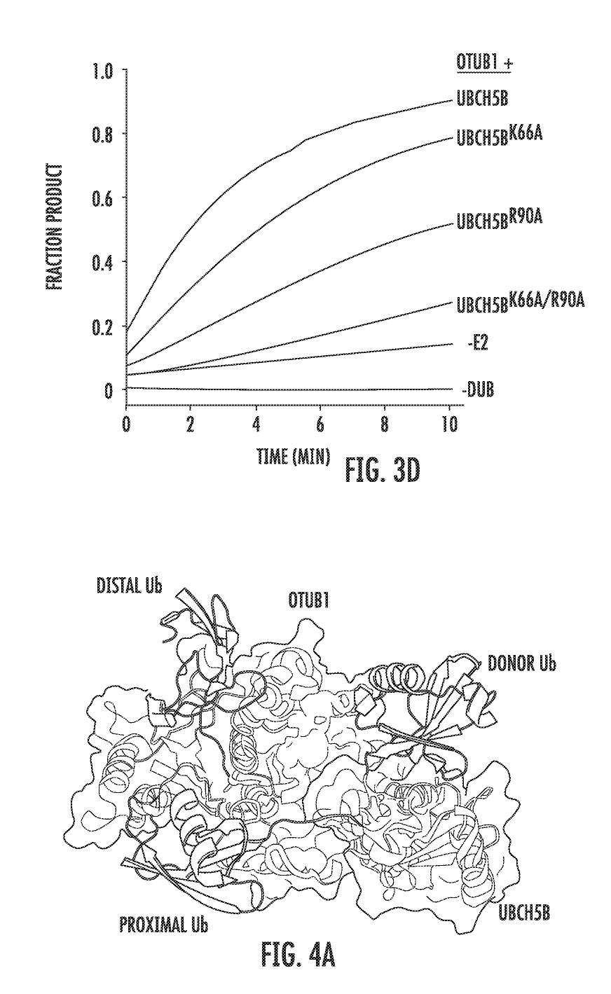

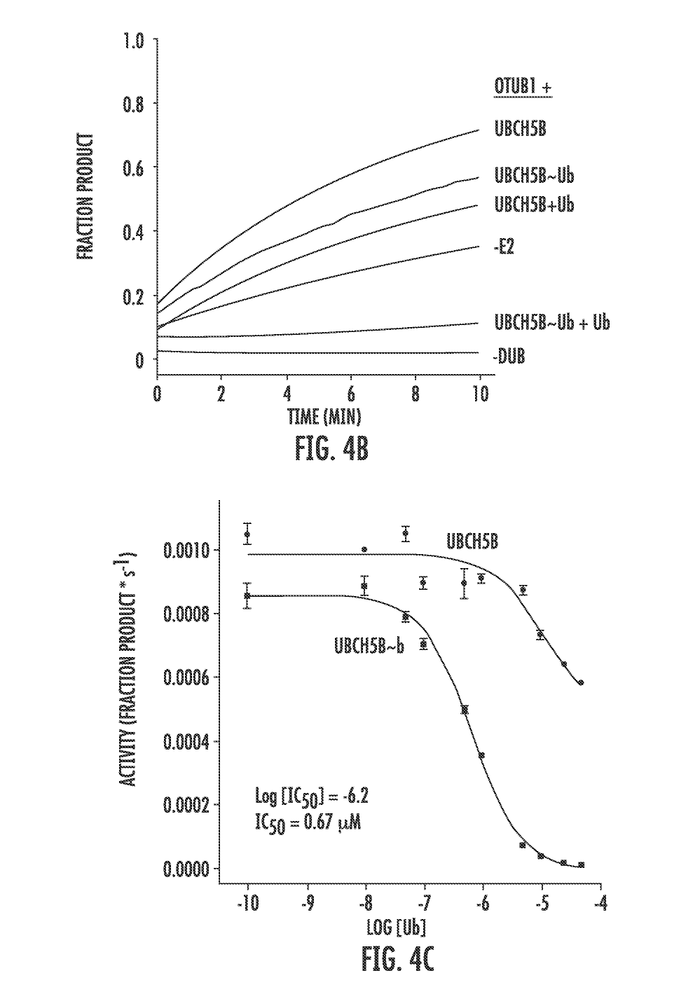

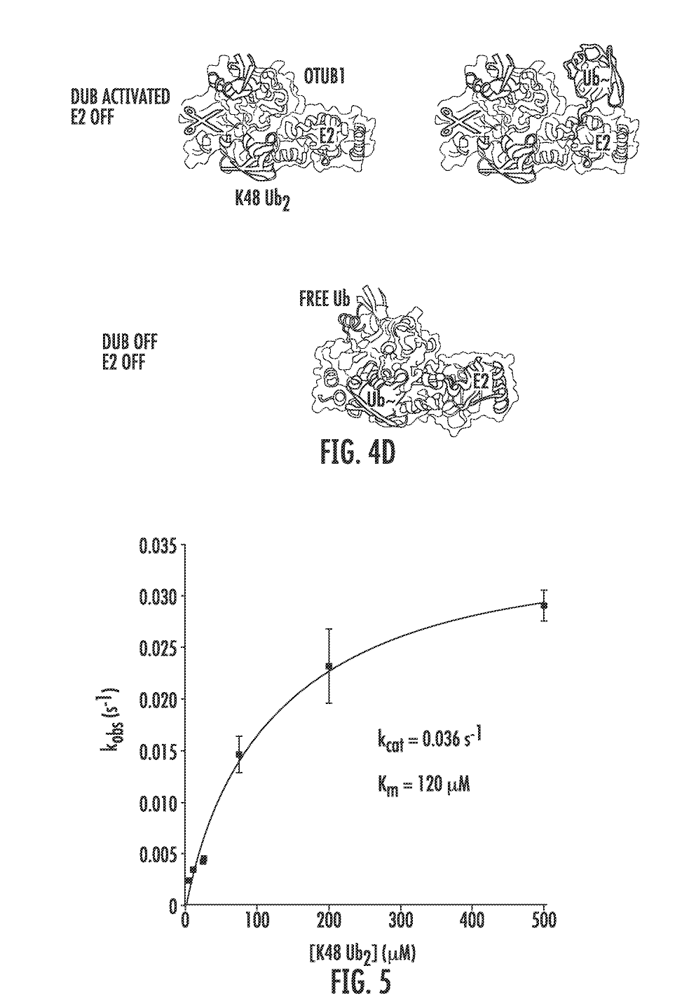

In accordance with one or more embodiments the present invention provides OTUB1-E2 complexes that can serve as molecular sensors that can toggle between cleaving K48 polyubiquitin to release free ubiquitin monomers and inhibiting ubiquitination by a subset of polyubiquitinating (UBCH5A/B/C, UBC13) and monoubiquitinating (UBE2W, UBCH6) E2 enzymes. Whether OTUB1-E2 complexes act as DUBs or inhibitors of polyubiquitination would thus depend upon the relative proportion of charged E2 enzymes and the local concentration of free ubiquitin and K48 polyubiquitin (FIG. 4D).

Since both E2 inhibition and OTUB1 stimulation depend upon a common OTUB1-E2 interface, the methods of the present invention can be exploited to carry out high throughput screens for small molecules that disrupt either function of OTUB1-E2 pairs by screening for small molecules that disrupt E2-stimulated cleavage of K48 diubiquitin substrates.

In accordance with an embodiment, the present invention provides a method for screening compounds which modulate the activity of an E2 peptide on the isopeptidase activity of OTUB1 comprising: a) providing a solution comprising a sufficient amount of K48 UB2 OTUB1 enzyme substrate; b) adding to the solution of a) a known quantity of an E2 enzyme; c) adding to the solution of a) a known quantity of a target compound; d) contacting the solution of c) with a known amount of OTUB1 enzyme for a specified period of time; to allow the enzymatic deubiquination of the K48 UB2 by OTUB1; e) quenching the deubiquination reaction of d); f) separating the resultant reaction products from the reaction of d); g) quantifying the amount of deubiquination of the K48 UB2 and comparing the amount of deubiquination to a control solution without the E2 enzyme and a positive control solution without the target compound; and g) determining whether the amount of deubiquination was increased or decreased relative to the amount of deubiquination in the control solution and whether the amount of deubiquination was increased or decreased relative to the amount of deubiquination in the positive control solution.

As used herein, the term “OTUB1” is synonymous with Otubain-1, and is a member of the OTU (ovarian tumor) superfamily of predicted cysteine proteases. The encoded protein is a highly specific ubiquitin iso-peptidase, and cleaves ubiquitin from branched poly-ubiquitin chains, being specific for lysine⁴⁸-linked polyubiquitin but not lysine⁶³-linked polyubiquitin.

“E2 enzymes” are also known as “Ubiquitin-conjugating enzyme, E2.” The activated ubiquitin linked to an E1 enzyme, is then transferred to an E2 cysteine. Once conjugated to ubiquitin, the E2 molecule binds one of several ubiquitin ligases or E3s via a structurally conserved binding region. In accordance with one or more embodiments, the methods of the present invention have shown that certain E2 enzymes stimulate deubiquination of the K48 UB2 complex, while others inhibit it. Those that stimulate deubiquination are referred to as “positive” or “stimulatory” E2 enzymes and those which inhibit deubiquination are referred to as “negative” or “inhibitory” E2 enzymes.

Examples of positive E2 enzymes include UBCH5A, UBCH5B, UBCH5C, UBE2W and UBCH6. Examples of negative E2 enzymes include UBC13, UBCH7, CDC34A, UBE2G2, RAD6A, and RAD6B.

The term “reagent kit,” or “test kit,” refers to an assembly of materials that are used in performing an assay. The reagents can be provided in packaged combination in the same or in separate containers, depending on their cross-reactivities and stabilities, and in liquid or in lyophilized form. The amounts and proportions of reagents provided in the kit can be selected so as to provide optimum results for a particular application.

A reagent kit embodying the features of the present invention comprises the K48 UB2 complex, E2 enzymes, buffers, quenchers, fluorescent labeled substrates, etc.

The phrase “calibration and control materials” refers to any standard or reference material containing a known amount of a drug to be measured. The concentration of drug is calculated by comparing the results obtained for the unknown specimen with the results obtained for the standard. This is commonly done by constructing a calibration curve.

The term “reacting” in the context of the embodiments of the present invention means placing compounds or reactants in proximity to each other, such as in solution, in order for a chemical reaction to occur between the reactants.

The invention further encompasses screening methods to identify derivatives and analogues of E2 enzymes which modulate the activity of OTUB1 as potential therapeutics for ubiquination related diseases and disorders.

The invention further provides for a method for screening for agents useful in the treatment of a disease or disorder associated with ubiquination comprising providing K48 UB2 complex, OTUB1 and an E2 enzyme or target compound and using these proteins in in vitro deubiquitination assays. Agents would be screened for their effectiveness in inhibiting deubiquitination in vitro.

As used herein, the term “target compound” encompasses antibodies, antibody fragments, proteins, peptides, siRNAs, antagonists, agonists, compounds, or nucleotide constructs which modulate the deubiquination of the K48 UB2 complex in the assay.

In accordance with one or more embodiments, the assays of the methods of the present invention can be of any type which can elucidate the deubiquination of the K48 UB2 complex. Examples of such assays include, but are not limited to, gel-based, fluorescence based, or radionuclide based assays. In some embodiments, the methods of the present invention use SDS-PAGE based methods. In some embodiments, the methods of the present invention use FRET based assays.

### EXAMPLES

Cloning and mutagenesis. Cloning of human OTUB1 was performed as described previously (J. Mol. Biol. 386:1011-23 (2009)). The genes encoding the E2 enzymes were PCR amplified from a human cDNA library and cloned into a pET vector containing N-terminal His6-SUMO-2 tag (pETSUMO-2), the pETSUMO-2 vector of the Infusion ligase free cloning system (Clonetech Inc., USA). Use of the N-terminal His6 tag allowed efficient purification of the fusion proteins, while the SUMO-2 tag was used to enhance solubility and also enabled precise removal of the complete tag using the SENP2 protease (Structure, 12:1519-31 (2004)). UBCH5BC85S was cloned into the pET32a vector containing the Trx-His tag followed by a TEV protease cleavage site. All other mutants of OTUB1 and UBCH5B were generated by site-directed mutagenesis using the Quick Change mutagenesis kit (Stratagene Inc.) following the manufacturer's protocol. The hybrid h/ceOTUB1 was generated as described previously (Nature 483:618-22 (2012)). All hOTUB1 N-terminal deletions were generated using Infusion ligase-free cloning (Clonetech Inc., USA).

Protein expression and purification. All proteins were expressed in E. coli Rosetta2 (DE3) cells grown in LB medium. Cultures were inoculated using 1% (v/v) overnight saturated cultures and were grown at 37° C. to an OD₆₀₀ of 0.8. Proteins were induced at 16° C. overnight by addition of 0.2 mM isopropyl-3-D-thio-galactoside (IPTG). Cells were harvested by centrifugation (8000×g, 15 minutes) and either lysed immediately or stored at −80° C. for later use. OTUB1 enzymes and ubiquitin were purified as previously described. Deletions and mutants of human OTUB1 were purified using the same protocol as the WT proteins. Cell pellets containing expressed E2 proteins were resuspended in lysis buffer (40 mM Na phosphate pH 8.0, 500 mM NaCl, 25 mM imidazole, 10 mM β-ME) supplemented with 0.1 mM phenyl-methyl sulfonyl fluoride (PMSF), 5 mM MgCl₂, and DNaseI. Cells were lysed using a Microfluidizer (Microfluidics Inc., Waltham, Mass.). The lysate was centrifuged to remove cell debris and was subjected to immobilized metal affinity chromatography (IMAC) using 5 mL HisTrap columns (GE Biosciences, USA). The protein was eluted with a linear imidazole gradient. Fractions containing purified protein were pooled and dialyzed overnight at 4° C. against 20 mM Na phosphate pH 8.0, 300 mM NaCl, 5 mM (3-ME. TEV or the SENP2 protease was added to the protein pool to cleave off the Trx-His or the His-SUMO-2 tag, respectively. Cleaved protein was then subjected to a second round of IMAC and the flow-through containing the cleaved protein was collected. Proteins were further purified by gel filtration on a preparative Superdex 75 column (GE Healthcare), dialyzed into 20 mM HEPES, pH 7.5, 150 mM NaCl, and 1 mM DTT, concentrated, and stored at −80° C. Proteins for crystallization and enzyme assays were >98% pure as visualized on a Coomassie-stained gel.

Purification of OTUB1˜Uba1:UBCH5BC85S˜Ub ternary complex. The oxyester linked UBCH5B^{C85S}˜Ub conjugate was prepared as previously described 35. Hybrid h/ceOTUB1, Uba1, and UBCH5B^{C85S}˜Ub were mixed at a 1:2:2 molar ratio and incubated on ice for 45 minutes. The mixture was then loaded on an analytical Superdex 75 column (GE Healthcare) pre-equilibrated in 20 mM Tris, pH 7.6, 100 mM NaCl, and 2 mM DTT. The OTUB1˜Uba1/UBCH5B^{C85S}˜Ub ternary complex eluted as a single peak and was concentrated to 12 mg/ml and stored at −80° C.

Crystallization. Crystals of the h/ceOTUB1-Uba1/UBCH5B^{C85S}˜Ub complex were grown at 20° C. from a 1:1 mix of purified complex (12 mg/ml) and well solution containing 100 mM BIS-TRIS pH 6, 200 mM MgCl2, 22% PEG 3350. Crystals appeared in about 2 to 3 days, were cryoprotected by well solution with added 10% ethylene glycol and then flash frozen in liquid nitrogen.

Data Collection and Structure Determination. A 2.5 Å diffraction data set was initially collected using an in-house Rigaku FR-E rotating anode x-ray generator (Rigaku, Japan) and a Rigaku Saturn 944+CCD detector under standard cryogenic conditions followed by processing with HKL2000 (Macromol. Crystallog., Pt A 276:307-326 (1997)). The structure of the /ceOTUB1˜Uba1:UBCH5B^{C85S}˜Ub ternary complex was determined by molecular replacement using PHASER (J. App. Crystallog. 40:658-674 (2007)). The h/ceOTUB1˜Uba1 complex (PDB ID: 4DHZ), UBCH5B (PDB ID: 2ESK), and ubiquitin (PDB ID: 1UBQ) were used as search models for molecular replacement. The model was further refined with successive rounds of real-space refinement in COOT (Section D, Biol. Crystallog. 60:2126-32 (2004)) and reciprocal-space refinement using REFMAC in CCP4 39, 40. The 2.5 Å structure was refined to a final R and R_{free} of 25.36% and 32.43% respectively.

A second 1.9 Å data set was collected at the GM/CA-CAT beamline 23-ID-D/B at the Advanced Photon Source under standard cryogenic conditions and processed with HKL2000 36. A high resolution structure of the h/ceOTUB1˜Uba1:UBCH5B^{C85S}˜Ub ternary complex was determined by refining the initial 2.5 Å structure against the newly obtained 1.9 Å diffraction data using REFMAC (Section D, Biol. Crystallog. 67:235-42 (2011); Meth. Enzymol. 374:300-21 (2003)). The model was further refined with successive rounds of real-space refinement in COOT and reciprocal-space refinement using REFMAC and PHENIX (Acta Crystallog. Section D, Biol. Crystallog. 68:352-67 (2012)). The final 1.9 Å structure was refined to an R and R_{free} of 18.72% and 22.45% respectively. The refined solution of provided clear density for all 76 residues of both ubiquitins, all 148 residues of UBCH5B, and residues 19-251 and 254-275 of h/ceOTUB1.

Protein-protein interaction surfaces were analyzed using the PISA server at EBI (pdbe.org/PISA) and manually inspected using COOT and PYMOL (pymol.org). Figures were generated with PYMOL.

Gel-based assay for UBCH5B stimulation of OTUB1 isopeptidase activity. K48 Ub2 deubiquitination assays were performed at 37° C. in a reaction buffer containing 20 mM HEPES pH 7.5, 100 mM NaCl, 5 mM DTT. 0.5 μM hOTUB1 was mixed with 15 μM K48 Ub2, in the presence and absence of 25 μM UBCH5B. Reactions were initiated by the addition of the OTUB1 enzyme. Specified time points were removed and reactions were quenched by the addition of denaturing SDS-PAGE loading dye containing β-ME. Samples were analyzed by gel electrophoresis on 4-12% polyacrylamide Bis-Tris Criterion XT gels (Bio-Rad, USA). Gels were stained with Coomassie brilliant blue.

Gel based assay for OTUB1 steady-state kinetics. Steady-state enzyme kinetic assays were performed at 37° C. in a reaction buffer containing 20 mM HEPES pH 7.5, 100 mM NaCl, 5 mM DTT. Human OTUB1 (0.03 μM) was mixed with specified amounts of K48 Ub2, in the presence and absence of 10 μM UBCH5B. Aliquots of the reaction mixtures were removed at specified time points, quenched by the addition of denaturing SDS-PAGE loading dye containing β-ME, and analyzed by SDS-PAGE followed by staining with SYPRO Ruby protein stain (Life Technologies, USA). The concentration of the ubiquitin product band for each time point was quantified by densitometry using the ImageJ software (Schneider et al. Nature Methods, 2012). Reaction velocities were then determined for each K48 Ub2 concentration and fit to the Michaelis-Menten equation using the GraphPad Prism software (GraphPad Software, Inc., La Jolla, Calif.).

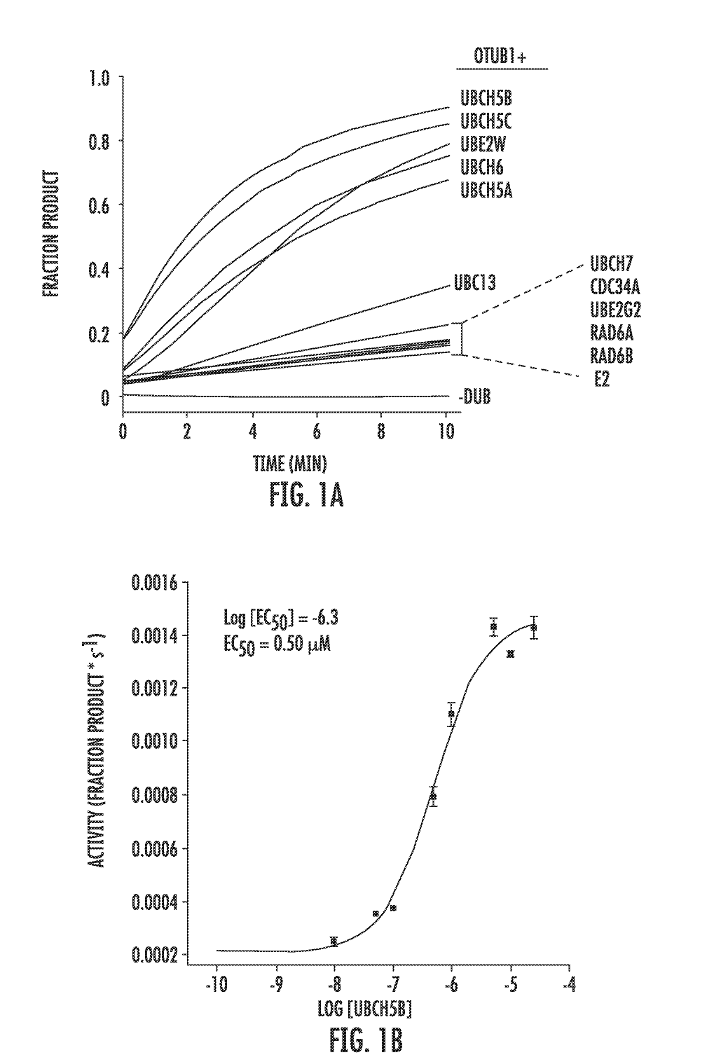

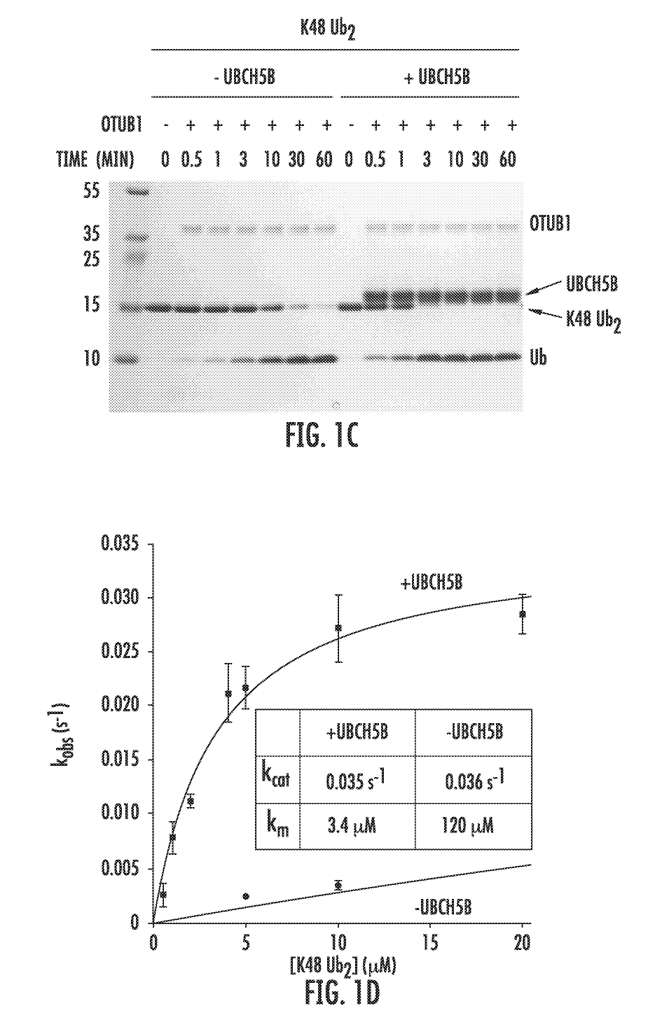

FRET-based assay for OTUB1 isopeptidase activity. All experiments were performed at 30° C. in buffer containing 20 mM HEPES pH 7.5, 100 mM NaCl, 5 mM DTT, 0.01% BSA and 0.4 μM of K48 Ub2 internally quenched fluorescent (IQF) substrate #5 (LifeSensors, Malvern, Pa.). Reactions were initiated by the addition of specified amounts of OTUB1. TAMRA fluorescence (ex. 544 nm, em. 590 nm) was monitored using a POLARStar Omega plate reader (BMG LABTECH). The initial rate of K48 Ub2 cleavage was calculated using the slope of the linear part of the fluorescent curves. For FIGS. 1b and 4c, nonlinear regression fitting in GraphPad Prism (GraphPad Software, Inc., La Jolla, Calif.) was used to analyze the data and fit the 50% stimulation/inhibition concentrations.

Ubiquitin-AMC assay for OTUB1 isopeptidase activity. All experiments were performed at 30° C. in a buffer containing 20 mM HEPES pH 7.5, 100 mM NaCl, 5 mM DTT, 0.01% BSA, and 10 μM of ubiquitin-AMC (Boston Biochem, Cambridge, Mass.). Reactions were initiated by the addition of 5 μM OTUB1 and carried out in the presence and absence of 10 μM UBCH5B. AMC fluorescence (ex. 380 nm, em. 460 nm) was monitored using a POLARStar Omega plate reader (BMG LABTECH).

Cell-based immunofluorescence experiments. Transfection, irradiation and immunostaining of HeLa cells was performed as described previously (Nat. Struct. Mol. Biol. 19:201-6 (2012)).

### Example 1

E2 enzymes stimulate OTUB1 K48 deubiquitinating activity. To test the effect that E2 enzymes might have on OTUB1 activity, the ability of OTUB1 to cleave K48-linked diubiquitin (K48 UB2) was measured in the presence of human E2 enzymes using a FRET-based assay. While it was initially expected that E2 enzymes would inhibit OTUB1 activity, it was unexpectedly found that five of the eleven E2s tested, UBCH5A-C, UBE2W and UBCH6, robustly stimulate K48-diubiquitin cleavage by OTUB1 (FIG. 1A). With the exception of UBE2W, these enzymes had previously been shown to interact with OTUB1 11. Surprisingly, UBC13, which is inhibited by OTUB1, only modestly stimulated OTUB1 as compared to the other activating E2s. OTUB1 stimulation was assayed as a function of UBCH5B concentration and found that half-maximal stimulation of OTUB1 cleavage activity occurred at 0.5 mM UBCH5B (FIG. 1B) (Table 1).

TABLE 1
UBCH5B EC50 value and statistics from fits to the Log(agonist)
vs. response model for K48 Ub2 (0.4 μM) cleavage
by OTUB1 (0.03 μM) in the presence of UBCH5B.

| Log(agonist) vs. response | |
|---|---|
| Analysis | |
| Best-fit values | |
| Bottom (fraction product * s⁻¹) | 0.00021 |
| Top (fraction product * s⁻¹) | 0.0015 |
| Log(EC₅₀) | −6.3 |
| EC₅₀ (M) | 5.0 × 10⁻⁷ |
| Span (fraction product * s⁻¹) | 0.0013 |
| 95% Confidence Intervals | |
| Bottom (fraction product * s⁻¹) | 0.00016 to 0.00026 |
| Top (fraction product * s⁻¹) | 0.0014 to 0.0015 |
| Log(EC₅₀) | −6.4 to −6.2 |
| EC₅₀ (M) | 3.9 × 10⁻⁷ to 6.4 × 10⁻⁷ |
| Span (fraction product * s⁻¹) | 0.0012 to 0.0013 |
| Goodness of Fit | |
| Degrees of Freedom | 24 |
| R² | 0.98 |
| Absolute Sum of Squares | 1.0 × 10⁻⁷ |
| Sy.x | 6.5 × 10⁻⁵ |
| Number of points | |
| Analyzed | 27 |

To confirm that the observed stimulation was not assay dependent, a gel-based assay was used to verify that UBCH5B stimulates OTUB1 cleavage of native K48-linked diubiquitin (FIG. 1C). In the absence of UBCH5B, OTUB1 consumed all of the diubiquitin substrate in 60 minutes, while OTUB1 processed the same amount of diubiquitin in 3 minutes when UBCH5B was present. To gain insight into which aspect of OTUB1 isopeptidase activity is stimulated, the steady-state kinetic parameters for K48 diubiquitin cleavage was determined by OTUB1 in the presence and absence of UBCH5B (FIGS. 1D; 5 and Table 2). It was found that UBCH5B reduced the K_{M} of OTUB1 for K48 diubiquitin by 35-fold (from 120 μM to 3.4 μM in the presence UBCH5B,), but had no effect on k_{cat} (0.035 s−1 and 0.036 s−1 in the presence and absence of UBCH5B, respectively). The relatively low k_{cat} for diubiquitin cleavage by OTUB1 allows for the interpretation of K_{M} as an approximation of the K_{d} of the OTUB1 for diubiquitin, therefore suggesting that UBCH5B stimulates OTUB1 by increasing its affinity for the K48 diubiquitin substrate.

TABLE 2
Kinetic constants and statistics from Michaelis-Menten fits
to steady-state saturation kinetics of K48 UB2 cleavage by OTUB1
(0.03 μM) in the presence and absence of UBCH5B (10 μM).

| | |
|---|---|
| Michaelis-Menten | Data Sets |

| Analysis | −UBCH5B | +UBCH5B |
|---|---|---|
| Best-fit values | | |
| k_{cat} (s⁻¹) | 0.036 | 0.035 |
| Kₘ (μM) | 120 | 3.4 |
| Std. Error | | |
| k_{cat} (s⁻¹) | 0.0032 | 0.0019 |
| Kₘ (μM) | 29 | 0.52 |
| 95% Confidence Intervals | | |
| k_{cat} (s⁻¹) | 0.030 to 0.043 | 0.031 to 0.039 |
| Kₘ (μM) | 58 to 180 | 2.3 to 4.5 |
| Goodness of Fit | | |
| Degrees of Freedom | 16 | 19 |
| R² | 0.94 | 0.95 |
| Absolute Sum of Squares | 0.00013 | 0.00010 |
| Sy.x | 0.0028 | 0.0023 |
| Constraints | | |
| Kₘ | Kₘ > 0.0 | Kₘ > 0.0 |
| Number of points | | |
| Analyzed | 18 | 21 |

### Example 2

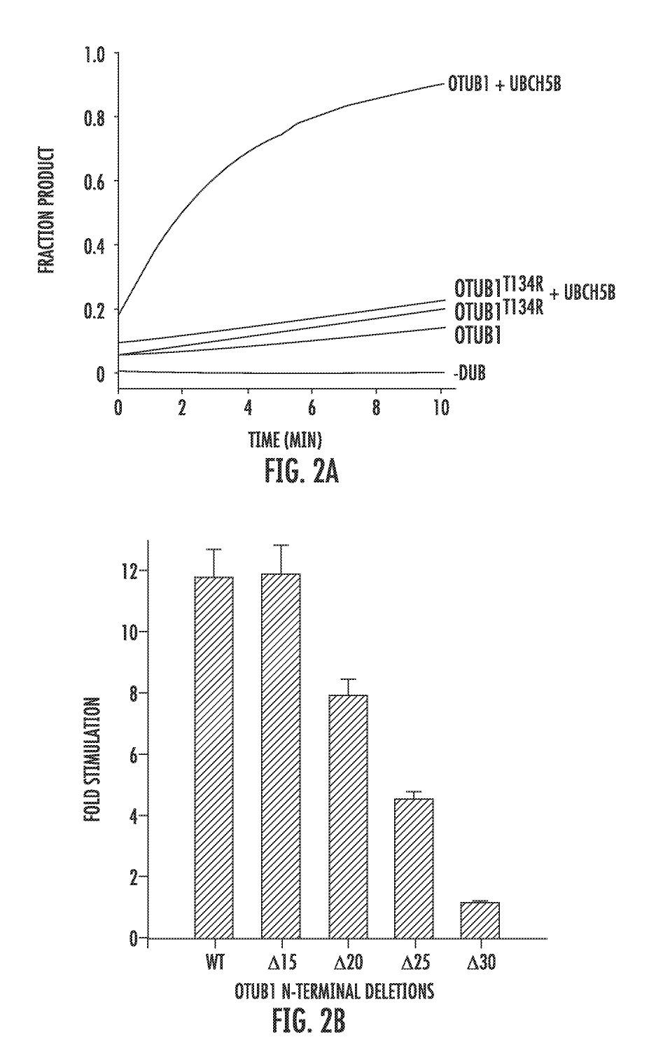

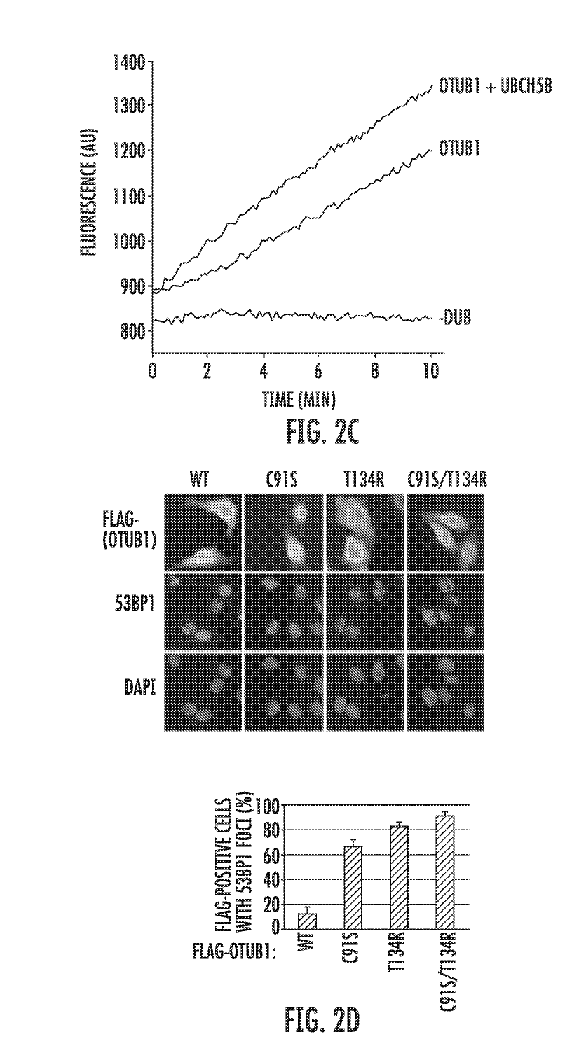

OTUB1 stimulation relies on the same set of E2 interactions as non-catalytic inhibition. To understand how OTUB1 binds UBCH5B, we first investigated whether the same interactions between OTUB1 and UBCH5B that are involved in E2 inhibition are also required for OTUB1 stimulation. A T134R substitution in the catalytic OTU domain of OTUB1 that disrupts binding to UBCH5B was previously shown to reduce OTUB1 inhibition of UBCH5B polyubiquitination. The ability of OTUB1-T134R to be stimulated by UBCH5B was assayed and found that this mutant was insensitive to UBCH5B, suggesting that the same OTUB1-E2 interface is needed for both stimulation and inhibition (FIG. 2A). Since the residues N-terminal to the OTU domain that forms the proximal ubiquitin-binding helix of OTUB1 play an essential role in E2 inhibition, we investigated whether E2 stimulation of OTUB1 activity similarly requires the OTUB1 N-terminus. As shown in FIG. 2B, deletion of the 15 N-terminal residues of OTUB1 did not affect its ability to be stimulated by UBCH5B, whereas deletion of the first 30 N-terminal residues completely disrupted stimulation. These results are consistent with a role for the OTUB1 N-terminal helix similar to that in the OTUB1-Ub-UBC13˜Ub inhibited complex, in which the first 23 residues are disordered while residues 24-44 form an ubiquitin-binding helix.

The importance of the OTUB1 N-terminal helix to its ability to be stimulated by the UBCH5B E2, together with its role in forming the proximal ubiquitin-binding site of OTUB1, suggested that UBCH5B increases OTUB1 affinity for K48 diubiquitin via the proximal ubiquitin-binding site. Based on this interpretation, it was hypothesized that UBCH5B would not stimulate OTUB1 cleavage of ubiquitin-AMC, a substrate that binds to the distal ubiquitin-binding site of OTUB1 but contains an isopeptide-linked fluorophore (amino methyl coumarin) in place of a proximal ubiquitin. Indeed, UBCH5B did not stimulate ubiquitin-AMC cleavage by OTUB1 (FIG. 2C), consistent with the idea that UCH5B increases the affinity of OTUB1 for the proximal ubiquitin in K48 diubiquitin but does not affect substrate binding to the distal ubiquitin-binding site.

### Example 3

OTUB1 catalytic activity and E2 interactions play a role in the DSB response. To evaluate the importance of OTUB1-E2 interactions to OTUB1 isopeptidase activity in vivo, we investigated whether a mutation affecting OTUB1-E2 interactions has an effect similar to that of a catalytic mutation. The effect of OTUB1 mutations on the formation of foci containing 53BP1 was therefore assayed, which together with BRCA1 controls the balance between DNA repair by non-homologues end joining versus homologous recombination. As shown in FIG. 2D, overexpression of wild type OTUB1 disrupts 53BP1 foci formation as previously reported, but neither the catalytically inactive OTUB1-C91S mutant or the OTUB1-T134R interface mutant disrupts foci formation. These results are consistent with a requirement that OTUB1 isopeptidase activity be stimulated by E2 enzymes in order to disrupt 53BP1 foci formation. We note that our finding that the OTUB1-C91S mutant did not inhibit 53BP1 foci formation like wild-type OTUB1 (FIG. 2D) differs from a previous report 11. It is possible that different ectopic expression levels between the two experiments or different times at which cells were fixed and imaged could account for the difference. We noted that very high expression of wild-type or mutant OTUB1 could cause nuclei to shrink and cells to undergo apoptosis. Therefore, only cells with normal morphology and relatively low levels of OTUB1 expression were included.

The effect of OTUB1 catalytic activity on 53BP1 recruitment indicates a role for cleavage of K48-linked polyubiquitin in order to accumulate 53BP1 at damage sites, which is RNF8 and RNF168-dependent. Although we do not yet know the identity of the K48-modified substrates, it is thought that OTUB1 may directly counteract RNF8-9 mediated K48-linked ubiquitination, which has been shown to facilitate the exposure of dimethylated Lys20 in histone H4 (H4K20me²), a docking site for 53BP1. Possible substrates include lysine demethylases and methyl lysine-binding proteins that are ubiquitinated upon DNA damage and regulate recruitment of 53BP1. It remains to be determined which factors upstream of 53BP1 are controlled by OTUB1 isopeptidase activity.

### Example 4

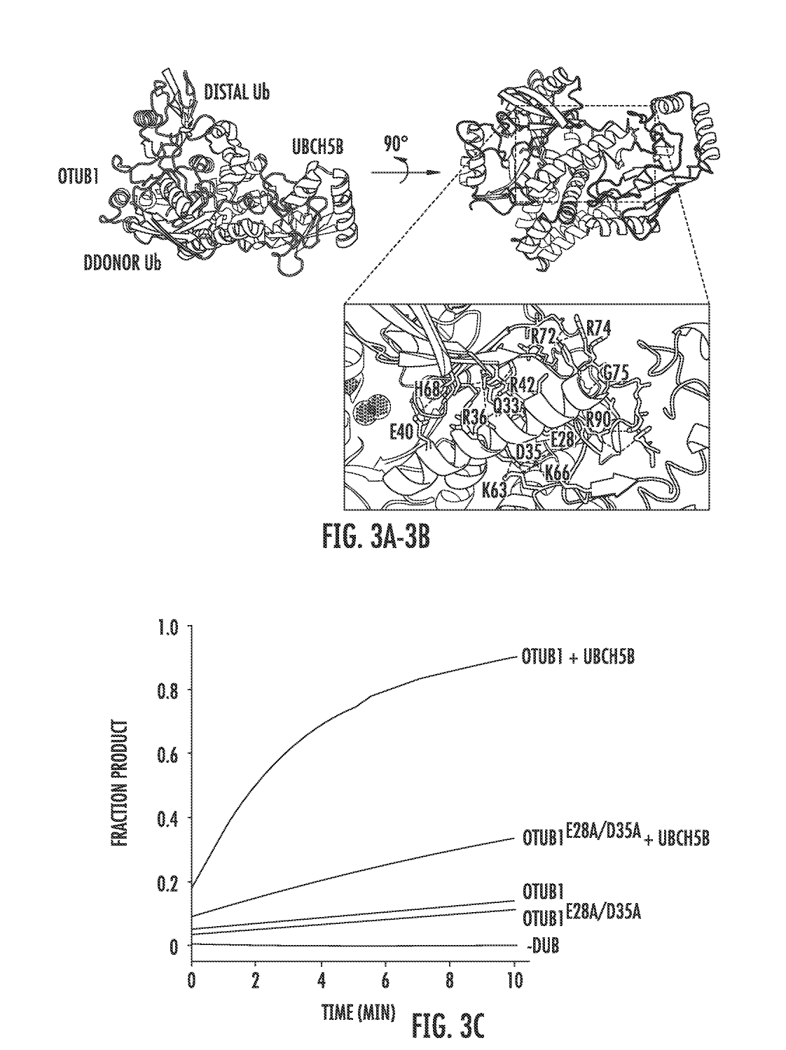

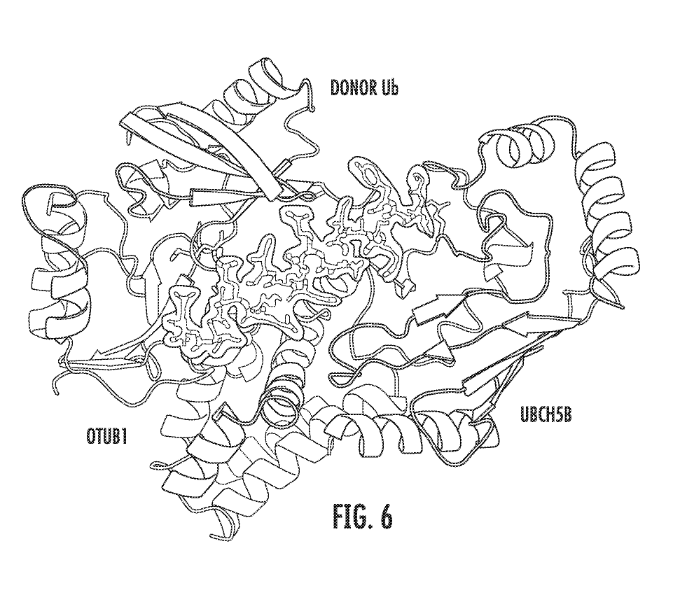

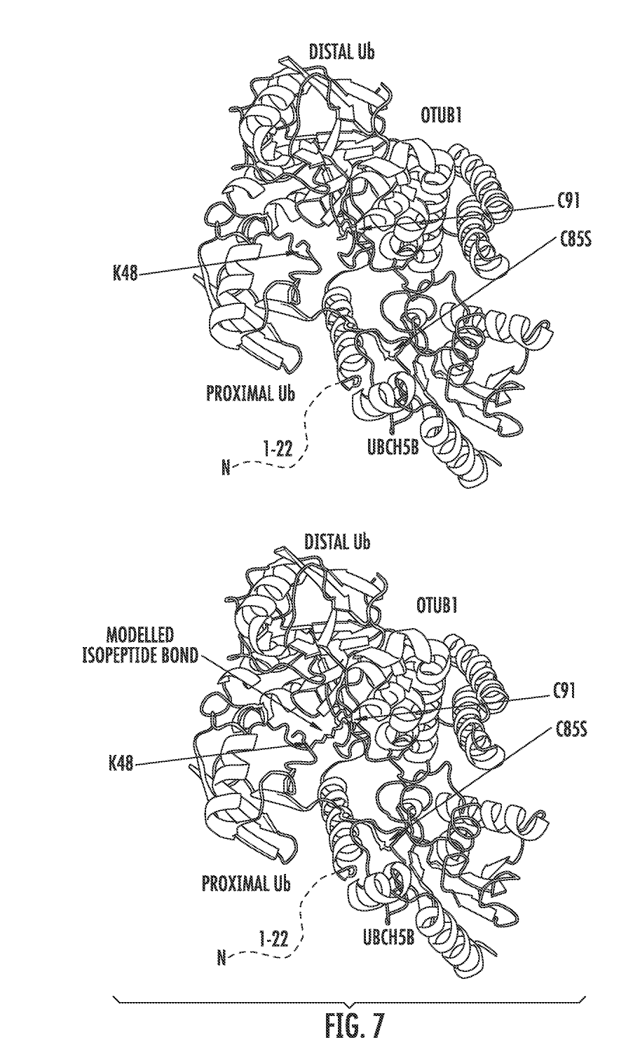

Structural basis for the role of UBCH5B in stabilizing the OTUB1 N-terminal helix. The present results suggest that UBCH5B stimulates binding of the K48-linked proximal ubiquitin to the OTUB1 N-terminal ubiquitin binding helix. These residues, which are N-terminal to the OTU catalytic domain, are disordered in the apoenzyme, but form an alpha helix when OTUB1 is bound to both an allosteric distal ubiquitin and charged UBC13˜Ub or UBCH5B˜Ub. In the absence of a structure of OTUB1 bound to K48 diubiquitin and E2, we took advantage of the observation that the two ubiquitins bound to the proximal and distal sites of OTUB1 mimic the binding of K48 diubiquitin to examine the structural effect of E2 binding on the N-terminal ubiquitin binding helix of OTUB1. The reported structures, however, do not show direct contacts between the E2 and the OTUB1 N-terminal arm, possibly due to limits in data resolution and order. In addition, the structure of the UBCH5B repression complex contains UBCH5B fused directly to the OTUB1 N-terminal helix, and thus does not address how a free OTUB1 N-terminus might contact UBCH5B. It was therefore determined a 1.9 Å resolution crystal structure of UBCH5B^{C85S}˜Ub bound to OTUB1 containing ubiquitin aldehyde (Uba1) bound to its distal site (data collection and refinement statistics shown in Table 3). The structure contains a hybrid OTUB1 containing the N-terminal 45 residues of human OTUB1 and the OTU domain of worm OTUB1, which was used in previous structural studies of the OTUB1-UBC13 repression complex. In the present structure, the N-terminal 22 residues of OTUB1 are disordered, while residues 23-44 (hOTUB1numbering) form a well-ordered alpha helix, which is consistent with the effect of N-terminal deletions on stimulation by UBCH5B (FIGS. 3A and 6). AK48 isopeptide linkage between the two ubiquitins can be readily modeled through minor adjustments of the proximal K48 side chain and the distal ubiquitin C-terminus, which is covalently linked to the active site cysteine in the structure (FIG. 7).

The structure of the OTUB1-Uba1:UBCH5B˜Ub complex reveals a network of direct and water-mediated contacts between UBCH5B and the N-terminal helix of OTUB1 that could stabilize folding of the OTUB1 N-terminus. OTUB1 residue Glu 24 is sandwiched between UBCH5B residues Lys 66 and Arg 90 and OTUB1-Glu31 hydrogen bonds with UBCH5B-Lys 63, while OTUB1-Leu 20 forms hydrophobic interactions with UBCH5Bleu 119 (FIG. 3A). In addition, there are extensive contacts between the C-terminal tail of the proximal ubiquitin and the OTUB1 N-terminal helix (FIG. 3A) that were not seen in previous structures. To test whether the observed contacts between the N-terminal helix of OTUB1 and UBCH5B are also important for stimulating OTUB1 cleavage of K48 diubiquitin, the effect of mutants designed to disrupt interactions between UBCH5B and the OTUB1 N-terminus without affecting contacts with the proximal ubiquitin were assayed. The OTUB1 double E35A/D28A substitution greatly decreased stimulation by 11 UBCH5B without affecting OTUB1 activity in the absence of UBCH5B (FIG. 3B). Similarly, UBCH5B mutations R90A or K66A, which disrupt interactions with Glu24 of OTUB1 (FIG. 3A), decreased the ability of UBCH5B to stimulate OTUB1, while the double K66A/R90A substitution in UBCH5B completely abolished its ability to stimulate OTUB1 (FIG. 3C). Taken together, the present results indicate that UBCH5B stimulates OTUB1 cleavage of K48 diubiquitin by buttressing the N-terminal helix of OTUB1, thereby stabilizing the proximal ubiquitin-binding site and raising the affinity of OTUB1 for K48 diubiquitin.

TABLE 3
Crystallographic data collection and refinement statistics
for OTUB1-Ubal:UBCH5B~Ub quaternary complex.

| | Home Source | GM/CA-CAT BL 23-ID-D/B | |
|---|---|---|---|
| | Cu—K_{α} | (Advanced Photon Source) | |
| Data collection | | | |
| Wavelength | 1.54 | 1.03 | |
| (Å) | | | |
| Space group | P22₁2₁ | P22₁2₁ | |
| Cell | | | |
| dimensions | | | |
| a, b, c (Å) | 46.9, 105.7, 130.9 | 46.5, 104.9, 131.6 | |
| α, β, γ (°) | 90, 90, 90 | 90, 90, 90 | |
| Resolution (Å) | 50.00-2.50 (2.54-2.50) | 50.00-1.90 | (1.93-1.90) |
| R_{sym} (%) | 10.70 (88.40) | 13.00 | (57.0) |
| Mean I/σ(I) | 17.82 (1.88) | 8.54 | (1.65) |
| Completeness | 99.42 (98.47) | 98.96 | (95.43) |
| (%) | | | |
| Multiplicity | 7.10 (7.00) | 8.00 | (3.10) |
| Refinement | | | |
| Resolution (Å) | | 1.90 | |
| Total | | 411948 | |
| reflections | | | |
| Unique | | 51047 | |
| reflections | | | |
| R_{work}/R_{free} | | 18.72/22.45 | (24.91/29.62) |
| No. atoms | | 5235 | |
| Protein | | 4552 | |
| Water | | 474 | |
| Ligands/ions | | 209 | |
| Protein | | 555 | |
| Residues | | | |
| Average | | 32.60 | |
| B-factor | | | |
| Protein | | 30.70 | |
| Solvent | | 41.70 | |
| R.M.S. | | | |
| deviations | | | |

| | |
|---|---|
| Bond lengths | 0.008 |
| (Å) | |
| Bond angles (°) | 1.20 |
| Ramachandran | 0 |
| outliers (%) | |
| Ramachandran | 98 |
| preferred (%) | |

### Example 5

The structural and biochemical studies presented herein show how E2 enzymes can stimulate the DUB activity of OTUB1. However, a significant proportion of E2 enzymes in the cell are charged with ubiquitin and there is also free unconjugated ubiquitin. Given the overlapping mechanisms of non-catalytic E2 inhibition and OTUB1 activation, we investigated whether OTUB1 can also be stimulated by charged UBCH5B˜Ub and whether the presence of free ubiquitin affects OTUB1 deubiquitinating activity. Model building based on the present work and the structure of charged UBCH5A˜Ub shows that OTUB1 in complex with K48 diubiquitin could bind to a charged UBCH5B˜Ub without any clashes between the additional ubiquitin and the diubiquitin occupying the OTUB1 proximal and distal sites (FIG. 4A). The ability of UBCH5B˜Ub to stimulate OTUB1 was assayed (FIG. 4B) and found that both charged and uncharged UBCH5B stimulate OTUB1 to a similar degree. However, assays of OTUB1 stimulation by UBCH5B or UBCH5B˜Ub over a range of ubiquitin concentrations (FIG. 4C; Table 4) showed that while stimulation by UBCH5B is relatively insensitive to free ubiquitin, stimulation by UBCH5B˜Ub decreases as a function of increasing concentration of free ubiquitin, dropping to low levels below ˜1 μM free ubiquitin. The complete repression of OTUB1 activity at >10 μM free ubiquitin is presumably due to formation of the inhibited OTUB1/Ub/UBCH5B˜Ub complex (FIG. 3A), which precludes binding of the K48 diubiquitin substrate.

TABLE 4
Free ubiquitin IC₅₀ value and statistics from fits to
the Log(inhibitor) vs. response model for K48 Ub2 (0.4
μM) cleavage by OTUB1 (0.03 μM).

| Log(inhibitor) vs. response | |
|---|---|
| Analysis | |
| Best-fit values | 3.6 × 10⁻⁵ |
| Bottom (fraction product * s⁻¹) | −1.25 × 10⁻⁵ |
| Top (fraction product * s⁻¹) | 0.00086 |
| Log(IC₅₀) | −6.17 |
| IC₅₀ (M) | 6.7 × 10⁻⁷ |
| Span (fraction product * s⁻¹) | 0.00087 |
| 95% Confidence Intervals | |
| Bottom (fraction product * s⁻¹) | −3.8 × 10⁻⁵ to 1.3 × 10⁻⁵ |
| Top (fraction product * s⁻¹) | 0.00083 to 0.00088 |
| Log(IC₅₀) | −6.3 to −6.1 |
| IC₅₀ (M) | 5.6 × 10⁻⁷ to 8.0 × 10⁻⁷ |
| Span (fraction product * s⁻¹) | 0.00084 to 0.00090 |
| Goodness of Fit | |
| Degrees of Freedom | 27 |
| R² | 0.99 |
| Absolute Sum of Squares | 3.4 × 10⁻⁵ |
| Sy.x | 3.6 × 10⁻⁵ |
| Number of points | |
| Analyzed | 30 |

Since the complex formed by OTUB1 with E2˜Ub and free ubiquitin (FIG. 3A) inhibits OTUB1 DUB activity as well as E2 activity, it is thought that the OTUB1-E2 “inhibited complex” may play an equally important role in regulating whether OTUB1 cleaves K48 polyubiquitin. The present results are consistent with a model in which OTUB1-E2 complexes can exist in three states (FIG. 4C), each with a different consequence for OTUB1 DUB activity. OTUB1 bound to an uncharged E2 actively cleaves K38 polyubiquitin, with the E2 lowering the KM for the K48 polyubiquitin substrate through interactions between the OTUB1 N-terminus and the E2 (FIG. 3A). At very low free ubiquitin concentrations, a charged E2˜Ub conjugate can similarly stimulate OTUB1, indicating that the neither the donor ubiquitin attached to the E2 nor free ubiquitin interferes with substrate binding under these conditions. At elevated ubiquitin concentrations, however, free ubiquitin binds to the OTUB1 distal site while the proximal site is occupied by the donor ubiquitin from the E2˜Ub conjugate. This configuration (FIG. 3A) precludes K48 diubiquitin substrate binding, thereby shutting down E2 stimulation and inhibiting diubiquitin cleavage, as well as inhibiting the E2. The different OTUB1-E2 complexes are in equilibrium with free OTUB1, which has lower isopeptidase activity, and free E2 and E2˜Ub, which are able to conjugate ubiquitin in concert with E1 and E3 enzymes. In vivo, the relative balance between different OTUB1 and E2 states will be determined by the proportion of free E2 to E2˜Ub thioester and the concentration of free ubiquitin. The switch between two opposing enzymatic activities provides a fuller explanation for how the previously observed ability of free ubiquitin to allosterically regulate OTUB1 non-catalytic inhibition and binding to E2˜Ub could be exploited to regulate ubiquitination in vivo. The availability of K48 polyubiquitin substrate could play an additional role in favoring active over repressed OTUB1.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for screening compounds which modulate the activity of an E2 peptide on the isopeptidase activity of OTUB1 comprising:
a) providing a solution comprising a sufficient amount of K48 UB2 OTUB1 enzyme substrate;
b) adding to the solution of a) a known quantity of an E2 enzyme;
c) adding to the solution of a) a known quantity of a target compound;
d) contacting the solution of c) with a known amount of OTUB1 enzyme for a specified period of time; to allow the enzymatic deubiquination of the K48 UB2 by OTUB1;
e) quenching the deubiquination reaction of d);
f) separating the resultant reaction products from the reaction of d);
g) quantifying the amount of deubiquination of the K48 UB2 and comparing the amount of deubiquination to a control solution without the E2 enzyme and a positive control solution without the target compound; and
g) determining whether the amount of deubiquination was increased or decreased relative to the amount of deubiquination in the control solution and whether the amount of deubiquination was increased or decreased relative to the amount of deubiquination in the positive control solution.

2. A method for measuring the modulatory activity of an E2 peptide on the isopeptidase activity of OTUB1 comprising:
a) providing a solution comprising a sufficient amount of K48 UB2 OTUB1 enzyme substrate;
b) adding to the solution of a) a known quantity of an E2 enzyme;
c) contacting the solution of b) with a known amount of OTUB1 enzyme for a specified period of time; to allow the enzymatic deubiquination of the K48 UB2 by OTUB1;
d) quenching the deubiquination reaction of c);
e) separating the resultant reaction products from the reaction of c);
f) quantifying the amount of deubiquination of the K48 UB2 and comparing the amount of deubiquination to a control solution without the E2 enzyme; and
g) determining whether the amount of deubiquination was increased or decreased relative to the amount of deubiquination in the control solution.

3. A method for measuring the isopeptidase activity of OTUB1 comprising:
a) providing a solution comprising a sufficient amount of K48 UB2 OTUB1 enzyme substrate;
b) contacting the solution of a) with a known amount of OTUB1 enzyme for a specified period of time; to allow the enzymatic deubiquination of the K48 UB2 by OTUB1;
c) quenching the deubiquination reaction of b);
d) separating the resultant reaction products from the reaction of b); and
e) quantifying the amount of deubiquination of the K48 UB2.

4. The method of claim 1, wherein the separation of the resultant reaction products from the reaction of b) is by SDS-PAGE.

5. The method of claim 4, wherein the E2 enzyme is selected from the group consisting of UBCH5A, UBCH5B, UBCH5C, UBE2W and UBCH6.

6. The method of claim 5, wherein the method further comprises the addition of a negative control wherein the negative control is an E2 enzyme known to inhibit deubiquination of the K48 UB2.

7. The method of claim 6, wherein the E2 enzyme is selected from the group consisting of UBC13, UBCH7, CDC34A, UBE2G2, RAD6A, and RAD6B.
